(19) United States
(12) Patent Application Publication
(10) Pub. No.: US 2016/0151253 A1
(43) Pub. Date: Jun. 2, 2016

(54) METHOD FOR CLEANING TOOTH SURFACE, COMPOSITION FOR CLEANING TOOTH SURFACE, AND METHOD FOR USING THE COMPOSITION

(71) Applicant: GC CORPORATION, Tokyo (JP)

(72) Inventors: Sayako HOTTA, Tokyo (JP); Takuya SATO, Tokyo (JP)

(21) Appl. No.: 14/943,469

(22) Filed: Nov. 17, 2015

(30) Foreign Application Priority Data
Nov. 27, 2014 (JP) 2014-239781

(51) Int. Cl. A61K 8/24; A61K 8/25; A61K 8/34; A61K 8/02; A61Q 11/00

(52) U.S. Cl. CPC A61K 8/24; A61K 8/0245; A61Q 11/00; A61K 8/345; A61K 8/25; A61K 2800/28; A61K 2800/412; A61K 2800/596

(57) ABSTRACT

Provided is a method for cleaning tooth surfaces including: applying a composition for cleaning tooth surfaces to tooth surfaces, the composition including: 1 to 20% by weight of a casein phosphopeptide-amorphous calcium phosphate complex and/or a casein phosphopeptide-amorphous calcium fluoride phosphate complex; 0.001 to 3% by weight of xylitol and/or sodium saccharin; 20 to 80% by weight of a wetting agent; 0.1 to 20% by weight of silica whose average particle size is no less than 0.5 μm and no more than 50 μm; and water as a main component of a reminder; and maintaining a state in which a part of the composition for cleaning tooth surfaces remains in a mouth.

### TECHNICAL FIELD

The present invention relates to a method for cleaning tooth surfaces, a composition for cleaning tooth surfaces used for the method for cleaning tooth surfaces, and a method for using the composition for cleaning tooth surfaces.

### BACKGROUND ART

As an enhancement method of tooth substances, a method of applying a fluorine compound to tooth surfaces to enhance tooth enamel and improve the acid resistance is generally carried out. There is another method of applying a composition containing a casein phosphopeptide-amorphous calcium phosphate (CPP-ACP) complex to tooth surfaces to enhance tooth substances. In addition, a casein phosphopeptide-amorphous calcium fluoride phosphate (CPP-ACFP) complex containing fluorine is effective to prevent dental caries (for example see JP 2002-500626 A, JP 2002-540129 A, JP2005-145952 A).

A casein phosphopeptide-amorphous calcium phosphate complex and a casein phosphopeptide-amorphous calcium fluoride phosphate complex are substances made from a complex of a peptide originated from a protein of milk (casein) and mineral (amorphous calcium phosphate). Their constituent elements include calcium and phosphorus which are mineral components of teeth. Thus they are effective for demineralization and remineralization of teeth by touching tooth surfaces. Thus, inventions have been made for making them remain on tooth surfaces as long as possible (for example, see JP 2010-047494 A and JP 2011-073983 A).

### SUMMARY OF THE INVENTION

### Problems to be Solved by the Invention

However, the conventional compositions and the using methods thereof have a problem that the compositions are troublesome to be used and the using methods are difficult to be accustomed, since in the methods, a composition containing a casein phosphopeptide-amorphous calcium phosphate complex and/or a casein phosphopeptide-amorphous calcium fluoride phosphate complex covers over tooth surfaces, and the composition needs to be used after the tooth surfaces are cleaned, for example after tooth brushing. In addition, even if a composition containing a casein phosphopeptide-amorphous calcium phosphate complex and/or a casein phosphopeptide-amorphous calcium fluoride phosphate complex is used as a dentifrice, its cleansing effect on tooth surfaces is low.

An object of the present invention is to provide a method for cleaning tooth surfaces which enables enhancement of tooth substances and cleaning of tooth surfaces at the same time.

In addition, another object of the present invention is to provide a composition for cleaning tooth surfaces which can be preferably used for the method for cleaning tooth surfaces.

Further, another object of the present invention is to provide a method for using the composition for cleaning tooth surfaces.

### Solution to Problem

According to one embodiment of the present invention, provided is a method for cleaning tooth surfaces including applying a composition for cleaning tooth surfaces to tooth surfaces, the composition including: 1 to 20% by weight of a casein phosphopeptide-amorphous calcium phosphate complex and/or a casein phosphopeptide-amorphous calcium fluoride phosphate complex; 0.001 to 3% by weight of xylitol and/or sodium saccharin; 20 to 80% by weight of a wetting agent; 0.1 to 20% by weight of silica whose average particle size is no less than 0.5 μm and no more than 50 μm; and water as a main component of a reminder, polishing the tooth surfaces where the composition for cleaning tooth surfaces is applied, removing an excess of the composition for cleaning tooth surfaces after the tooth surfaces are polished, without washing a mouth with water, and maintaining a state in which a part of the composition for cleaning tooth surfaces remains in the mouth.

According to another embodiment of the present invention, provided is a composition for cleaning tooth surfaces including: 1 to 20% by weight of a casein phosphopeptide-amorphous calcium phosphate complex and/or a casein phosphopeptide-amorphous calcium fluoride phosphate complex; 0.001 to 3% by weight of xylitol and/or sodium saccharin; 20 to 80% by weight of a wetting agent; 0.1 to 20% by weight of silica whose average particle size is no less than 0.5 μm and no more than 50 μm; and water as a main component of a reminder, the composition for cleaning tooth surfaces to be applied to tooth surfaces, used for polishing the tooth surfaces, an excess of which is removed after the tooth surfaces are polished without washing a mouth with water, and maintained in a state in which a part of which remains in the mouth.

According to another embodiment of the present invention, provided is a method for using a composition for cleaning tooth surfaces, the method including applying a composition for cleaning tooth surfaces to tooth surfaces, the composition including: 1 to 20% by weight of a casein phosphopeptide-amorphous calcium phosphate complex and/or a casein phosphopeptide-amorphous calcium fluoride phosphate complex; 0.001 to 3% by weight of xylitol and/or sodium saccharin; 20 to 80% by weight of a wetting agent; 0.1 to 20% by weight of silica whose average particle size is no less than 0.5 μm and no more than 50 μm; and water as a main component of a reminder, polishing the tooth surfaces with the composition, removing an excess of the composition after the tooth surfaces are polished, without washing a mouth with water, and maintaining the composition in a state in which a part of the composition remains in the mouth.

### Advantageous Effects of Invention

According to one embodiment of the present invention, provided is a method for cleaning tooth surfaces which enables enhancement of tooth substances and cleaning of tooth surfaces at the same time.

In addition, according to another embodiment of the present invention, provided is a composition for cleaning tooth surfaces which can be preferably used for the method for cleaning tooth surfaces.

Further, according to another embodiment of the present invention, provided is a method for using the composition for cleaning tooth surfaces.

### DESCRIPTION OF EMBODIMENTS

Hereinafter the embodiments of the present invention will be described. The present invention is not limited to the embodiments, and various substitutions can be added to the embodiments within the scope of the present invention.

In the embodiments of the present invention, one structural example of the composition for cleaning tooth surfaces will be explained.

The composition for cleaning tooth surfaces of the embodiments can include: 1 to 20% by weight of a casein phosphopeptide-amorphous calcium phosphate complex and/or a casein phosphopeptide-amorphous calcium fluoride phosphate complex; 0.001 to 3% by weight of xylitol and/or saccharin sodium; 20 to 80% by weight of a wetting agent; 0.1 to 20% by weight of silica whose average particle size is no less than 0.5 μm and no more than 50 μm; and water as the main component of the reminder.

The composition for cleaning tooth surfaces of the embodiments can include a casein phosphopeptide-amorphous calcium phosphate complex and/or a casein phosphopeptide-amorphous calcium fluoride phosphate complex. The casein phosphopeptide-amorphous calcium fluoride phosphate complex can be prepared for example by: mixing calcium, phosphopeptide, an inorganic phosphoric acid and optionally fluoride; filtering the obtained mixture; and drying the mixture (for example see JP 2002-500626 A).

The mixture ratio of the casein phosphopeptide-amorphous calcium phosphate complex and/or casein phosphopeptide-amorphous calcium fluoride phosphate complex is 1 to 20% by weight in the composition for cleaning tooth surfaces. If the mixture ratio is less than 1% by weight, the effect of enhancing tooth substances tends to be small, and if more than 20% by weight, the viscosity of the composition gets high and the operability tends to degrade. More preferable mixture ratio is 2 to 15% by weight.

The composition for cleaning tooth surfaces of the embodiments can include xylitol and/or sodium saccharin as a sweetener. The mixture ratio of xylitol and/or sodium saccharin in the composition for cleaning tooth surfaces is 0.001 to 3% by weight. If the mixture ratio is less than 0.001% by weight, the effect tends to be small, and if more than 3% by weight, the uncomfortable feeling of the users tends to be large. More preferable mixture ratio is 0.001 to 1.5% by weight. The composition can also include a sweetener other than xylitol and/or sodium saccharin.

The composition for cleaning tooth surfaces of the embodiments can include a wetting agent. Examples of the wetting agent include glycerin, propylene glycol, sorbitol, mannitol, ethylene glycol, polyethylene glycol, polypropylene glycol, chondroitin sulfate, and salts thereof. Specifically, in view of making the users feel less uncomfortable, glycerin, propylene glycol and sorbitol are preferable.

The mixture ratio of the wetting agent in the composition for cleaning tooth surfaces is 20 to 80% by weight. If the mixture ratio is less than 20% by weight, the effect of wetting tends to be small, and if more than 80% by weight, the uncomfortable feeling of the users tends to be large. More preferable mixture ratio is 25 to 50% by weight.

The composition for cleaning tooth surfaces of the embodiments can include silica of no less than 0.5 μm and no more than 50 μm in average particle size, as a polishing component. If the average particle size is less than 0.5 μm, the polishing effect tends to be small, and if more than 50 μm, the preservation stability tends to degrade. The average particle size is more preferably no less than 1 μm and no more than 15 μm. The kind of the silica is not particularly limited, and known silica can be used, such as silica fine powder, amorphous hydrated silica, hydrophobic silica, and fumed silica. These kinds of silica can be used alone or in combination. It is noted that polishing components other than silica are not preferably used.

Examples of the silica of no less than 0.5 μm and no more than 50 μm in average particle size include wet silica and dry silica. Wet silica is more preferable since the amount of silanol groups on the surface thereof is relatively larger. Examples of wet silica include precipitated silica and gel-type silica, and both can be preferably used. Examples of commercialized products of precipitated silica include Sylopure (registered trademark, manufactured by FUJI SILYSIA CHEMICAL LTD.) and Nipsil (registered trademark, manufactured by Tosoh Silica Corporation). Examples of commercialized products of wet silica include NIPGEL (registered trademark, manufactured by Tosoh Silica Corporation).

As for the silica of no less than 0.5 μm and no more than 50 μm in average particle size, it is further preferable that the oil absorption of the silica is 50 to 500 ml/100 g. Here, the oil absorption of silica means the amount of oil that silica can hold, and the measurement method thereof is based on JIS K5101. The oil absorption of the silica within the above range, which can achieve an appropriate polishing power, is preferable. More preferably the oil absorption of the silica is 50 to 300 ml/100 g.

The mixture ratio of the silica of no less than 0.5 μm and no more than 50 μm in average particle size in the composition for cleaning tooth surfaces is 0.1 to 20% by weight. If the mixture ratio is less than 0.1% by weight, the polishing effect tends to be small, and if more than 20% by weight, the operability tends to degrade. More preferable mixture ratio of the silica of no less than 0.5 μm and no more than 50 μm in average particle size is 1 to 10% by weight.

The composition for cleaning tooth surfaces of the embodiments can further include fine particle silica of no less than 0.005 μm and less than 0.5 μm in average particle size. Using the fine particle silica of no less than 0.005 μm and less than 0.5 μm in average particle size in combination with the silica of no less than 0.5 μm and no more than 50 μm in average particle size provides an effect of further increasing the cleaning performance of the composition. Examples of the fine particle silica of no less than 0.005 μm and less than 0.5 μm in average particle size include AEROSIL (registered trademark, manufactured by NIPPON AEROSIL CO., LTD.).

The mixture ratio of the fine particle silica of no less than 0.005 μm and less than 0.5 μm in average particle size is preferably 0.1 to 15% by weight in the composition for cleaning tooth surfaces. The mixture ratio is further preferably 1 to 5% by weight.

The composition for cleaning tooth surfaces of the embodiments can further include a surfactant as a foaming agent. A surfactant has a foaming function and a function of helping the components to spread. The examples of the surfactant include sodium lauryl sulfate, sodium lauroylsarcosinate, sodium lauroylmethyltaurate, laurylbetaine, sodium cetyl sulfate, sodium lauryl ether sulfate, ammonium lauryl sulfosuccinate, ammonium lauryl sulfate, ammonium lauryl ether sulfate, sodium lauroyl isethionate, sodium dodecylbenzene sulfonate, triethanolamine dodecylbenzene sulfonate, and sodium cocoyl isethionate. These surfactants can be used alone or in combination.

The mixture ratio of the surfactant in the composition for cleaning tooth surfaces is preferably 0.1 to 3% by weight.

The composition for cleaning tooth surfaces of the embodiments can further include a thickening agent. By including a thickening agent, it is possible to adjust the viscosity such that the components of the composition are easy to attach to tooth surfaces. Examples of the thickening agent include: salts of carboxymethyl cellulose such as sodium carboxymethyl cellulose; natural products such as guar gum, carob bean gum, tara gum, tamarind seed gum, gum arabic, tragacanth gum, karaya gum, carrageenan, xanthan gum, gellan gum, curdlan, chitin, chitosan, and chitosamine; sodium alginate; propylene glycol alginate ester; sodium starch glycolate; sodium starch phosphate ester; sodium polyacrylate; and polyvinylpyrrolidone. These thickening agents can be used alone or in combination.

The viscosity of the composition for cleaning tooth surfaces of the embodiments is preferably 10 to 5000 Pa·s, and more preferably 200 to 2000 Pa·s, at 25° C. Thus, the mixture ratio of the thickening agent in the composition is preferably 0.5 to 10% by weight and more preferably 0.5 to 5% by weight.

The composition for cleaning tooth surfaces of the embodiments can further include a fluorine compound. By including a fluorine compound, the composition supplies fluorine to tooth substances, and the resistance of the teeth against dental caries increases. Examples of fluorine compound include sodium fluoride, tin fluoride, sodium monofluorophosphate, calcium fluoride, potassium fluoride, aluminum fluoride, sodium hexafluorosilicate, sodium fluorosilicate, ammonium fluorosilicate, amine fluoride, and ammonium fluoride. These fluorine compounds can be used alone or in combination.

The mixture ratio of the fluorine compound in the composition for cleaning tooth surfaces is preferably 0.01 to 5% by weight, and especially preferably 0.05 to 0.2% by weight. Each preferable mixture ratio in a case where only one kind of fluorine compound is mixed is, sodium fluoride: 0.011 to 1.10% by weight, tin fluoride: 0.02 to 2.0% by weight, sodium monofluorophosphate: 0.037 to 3.7% by weight. In terms of atomic weight of fluorine, the ratio is preferably 0.005 to 0.5% by weight.

The pH of the composition for cleaning tooth surfaces is preferably within the range of 5.5 to 9.5. As a way of adjusting the pH, mixing a solution of phosphoric acid or salt thereof as a pH adjuster can be given.

The composition for cleaning tooth surfaces of the embodiments can further include antibacterial agents such as titanium dioxide, coloring agents, preservatives, antifungal agents, flavoring agents and the like.

The reminder of the composition for cleaning tooth surfaces of the embodiments is mostly water. The mixture ratio of water in the composition is preferably 5 to 70% by weight.

One structural example of the method for cleaning tooth surfaces of the embodiments will be explained. In the method for cleaning tooth surfaces of the embodiments, the composition for cleaning tooth surfaces described above is used.

In the method for cleaning tooth surfaces of the embodiments, firstly, the composition for cleaning tooth surfaces is applied to tooth surfaces. The term “apply” includes “coat” for example. The method for applying the composition is not limited, and a method of using a brush such as a toothbrush can be given as an example.

Next, the tooth surfaces are polished with the composition for cleaning tooth surfaces applied thereto, and attached substances on the tooth surfaces such as food residues and dental plaque are physically removed. This can be done in the same way as in normal tooth brushing. Since the composition includes silica of no less than 0.5 μm and no more than 50 μm in average particle size as a polishing component, even attached substances, at the part where the brush does not reach, such as a gap between adjacent teeth, can be effectively removed.

Next, after the tooth surfaces are polished, excess fluid including the composition for cleaning tooth surfaces and attached substances on the tooth surfaces in the oral cavity is removed, for example by spitting. At this time, preferably washing mouth with water (gargle) as in normal tooth brushing is not carried out. Without washing mouth with water, a part of the composition for cleaning tooth surfaces remains in the oral cavity.

Next, the state that a part of the composition for cleaning tooth surfaces remains in the oral cavity is maintained. This makes the composition attach to the tooth surfaces, and the components of the composition continue to be supplied to the tooth surfaces. In specific, calcium and phosphorus are supplied to the tooth substances, from the casein phosphopeptide-amorphous calcium phosphate complex and/or the casein phosphopeptide-amorphous calcium fluoride phosphate complex, which promotes remineralization. In addition, fluorine is supplied to the tooth substances from the casein phosphopeptide-amorphous calcium fluoride phosphate complex, which results in increase of the resistance against dental caries. These effects make the tooth substances strong.

The time to maintain the state that a part of the composition for cleaning tooth surfaces remains in the oral cavity is not particularly limited. In view of reinforcement of the tooth substances, the time is preferably for 30 minutes to 2 hours.

Hereinafter the present invention will be described in more detail referring to examples. However, the present invention is not limited to these examples.

### EXAMPLES

### Preparation of Composition for Cleaning Tooth Surfaces

Each component was mixed having the ratio shown in Table 1, thereby the compositions for cleaning tooth surfaces of Examples 1 to 6 and Comparative Examples 1 to 3 were prepared. The unit of each component in Table 1 was % by weight, and the blanks mean that the corresponding component was not added. The average particle sizes and the oil adsorptions of silica are shown together.

### <Evaluation of Remineralization Effect>

Enamel blocks were made from a cow tooth sliced in approx. 1 mm in thickness, coated by manicure, on which 1-square-mm open space where manicure was not applied was left for examination. The enamel blocks were immersed in a demineralizing solution for 4 days under a 37° C. environment, whereby artificial caries were made. Each enamel block on which artificial caries were made was subject to the following pH cycle (1 day) of (1) to (4), and the cycle was repeated for 7 times (7 days).

(1) 5-minute immersion in a ten-fold dilute solution of the composition for cleaning tooth surfaces of Examples and Comparative Examples
(2) 6-hour immersion in a remineralizing solution
(3) 5-minute immersion in a ten-fold dilute solution of the composition for cleaning tooth surfaces of Examples and Comparative Examples
(4) approx. 18-hour immersion in a demineralizing solution
Composition of remineralizing solution (pH 7)
HEPES buffer salt: 20 mM
Potassium chloride: 130 mM
Calcium chloride: 1.5 mM
Potassium dihydrogenphosphate: 0.9 mM
Composition of demineralizing solution (pH 4.8)
Lactic acid: 0.1 M
Calcium chloride: 1.5 mM
Potassium dihydrogenphosphate: 0.9 mM
Sodium carboxymethyl cellulose: 1% by weight

X-ray images of the enamel block after the pH cycles were taken. Amounts of mineral at the demineralized part and the remineralized part were calculated, from the distance of the parts from the surface and the brightness of the parts, by means of an image analyzer. Whereby, the remineralization ratio was obtained. The larger the remineralization ratio was, the larger the degree of remineralization was. The evaluation was carried out with the following indicators. The results are shown in Table 1.

∘: having no less than 15% of remineralization ratio
Δ: having no less than 1% and less than 15% of remineralization ratio
x: having less than 1% of remineralization ratio

### <Evaluation of Cleansing Effect>

The evaluation was carried out based on the method described in Duncan Yu. “Stain removal efficacy of two new fluoride dentifrices containing essential oils” American Journal of Dentistry, (13), 23-25 (2000). Enamel of a cow tooth was embedded in a resin and the surface to be examined was polished. The specimen was subject to an acid treatment, thereafter immersed in a colored medium. Further, a Kocuria rhizophila culture solution in an amount of 1/20 of the colored medium and 0.2% iron (III) chloride aqueous solution were added to the medium, and shaking culture was carried out at 37° C. for 4 days. The specimen was taken out from the culture solution, thereafter subject to autoclave (121° C. for 15 minutes), to be a specimen for the tooth surface cleansing effect examination. The colored medium was prepared from 100 mL of TSB liquid medium, to which 0.34 g of instant coffee, 0.25 g of instant tea and 0.25 g of mucin were added, and which was subject to autoclave.

After the Lab value of the specimen was measured by a colorimeter, the specimen was immersed in a 40% by weight aqueous solution of the composition for cleaning tooth surfaces of Examples and Comparative Examples. The tooth surfaces were brushed with the brush of an abrasion tester, under a condition of 150 g of load for 800 strokes. After the examination, the Lab value of the specimen was measured, and ΔE (=√(ΔL²+Δa²+Δb²)) was calculated. The larger ΔE was, the higher the cleansing effect was. The evaluation was carried out with the following indicators. The results are shown in Table 1.

∘: ΔE is no less than 6
Δ: ΔE is no less than 2 and less than 6
x: ΔE is less than 2

TABLE 1

| | | (Unit: % by weight) | | | | | | | | |
|---|---|---|---|---|---|---|---|---|---|---|
| | | Ex. 1 | Ex. 2 | Ex. 3 | Ex. 4 | Ex. 5 | Ex. 6 | Comp. 1 | Comp. 2 | Comp. 3 |
| CPP-ACP complex and/or | CPP-ACP | 10 | 10 | 10 | 10 | 10 | 10 | 10 | | 10 |
| CPP-ACFP complex | | | | | | | | | | |
| silica | precipitated silica 1 | | | 4 | | | | | | |
| (average particle | (average particle size: | | | | | | | | | |
| size: no less than 0.5 | 11.5 μm) | | | | | | | | | |
| μm and no more than 50 | precipitated silica 2 | | | | | 2 | | | | |
| μm) | (average particle size: | | | | | | | | | |
| | 2.9 μm) | | | | | | | | | |
| | gel-type silica 1 | | 1 | | 1 | | | | 2 | |
| | (average particle size: | | | | | | | | | |
| | 2.2 μm, | | | | | | | | | |
| | average oil adsorption: | | | | | | | | | |
| | 80-130 ml/100 g) | | | | | | | | | |
| | gel-type silica 2 | 2 | | | | | 2 | | | |
| | (average particle size: | | | | | | | | | |
| | 4 μm, | | | | | | | | | |
| | average oil adsorption: | | | | | | | | | |
| | 105 ml/100 g) | | | | | | | | | |
| fine particle silica | fine particle silica | 2 | 4 | 3 | 4 | | | 2 | 2 | |
| (average particle | (average partilce size: | | | | | | | | | |
| size: no less than | 0.012 μm) | | | | | | | | | |
| 0.005 μm and less than | | | | | | | | | | |
| 0.5 μm) | | | | | | | | | | |
| xylitol and/or | xylitol | 1 | 1 | 1 | 1 | 1 | 1 | 1 | 1 | 1 |
| sodium saccharin | | | | | | | | | | |
| wetting agent | glycerin | 20 | 18 | 18 | 18 | 18 | 18 | 20 | 20 | 18 |
| | propylene glycol | 2 | 2 | 2 | 2 | 2 | 2 | 2 | 2 | 2 |
| | sorbitol | 24.5 | 24.5 | 24.5 | 24.5 | 10 | 10 | 3.5 | 20 | 10 |
| surfactant | sodium lauryl sulfate | | | 0.6 | | | | | | |
| | sodium lauroylsarcosinate | | | | 0.3 | | | | | |
| | sodium lauroylmethyltaurate | | | | | | | | 1 | |
| | laurylbetaine | | | | | 0.6 | 0.6 | | | 0.6 |
| thickening agent | sodium carboxymethyl | 1.9 | 2 | 2 | 3 | 2 | 2 | 2 | 2 | 2 |
| | cellulose | | | | | | | | | |
| fluorine compound | sodium fluoride | 0.32 | 0.2 | 0.2 | 0.2 | 0.2 | 0.2 | 0.2 | | |
| pH adjuster | phosphoric acid | 0.3 | 0.3 | 0.3 | 0.3 | 0.3 | 0.3 | 0.3 | 0.3 | 0.3 |
| antibacterial agent | titanium dioxide | 0.6 | 1 | 1 | 1 | 1 | 1 | 1 | 1 | 1 |

| | | | | | | | | | |
|---|---|---|---|---|---|---|---|---|---|
| flavoring agent | 0.2 | 0.2 | 0.3 | 0.2 | 0.2 | 0.2 | 0.2 | 0.2 | 0.2 |
| water | 35.18 | 35.8 | 33.1 | 34.5 | 52.7 | 52.7 | 57.8 | 48.5 | 54.9 |
| remineralization effect | ∘ | ∘ | ∘ | ∘ | ∘ | ∘ | ∘ | x | Δ |
| cleansing effect | ∘ | ∘ | Δ | ∘ | Δ | ∘ | x | ∘ | x |

1. A method for cleaning tooth surfaces comprising:
applying a composition for cleaning tooth surfaces to tooth surfaces, the composition comprising:
1 to 20% by weight of a casein phosphopeptide-amorphous calcium phosphate complex and/or a casein phosphopeptide-amorphous calcium fluoride phosphate complex;
0.001 to 3% by weight of xylitol and/or sodium saccharin;
20 to 80% by weight of a wetting agent;
0.1 to 20% by weight of silica whose average particle size is no less than 0.5 μm and no more than 50 μm; and
water as a main component of a reminder,
polishing the tooth surfaces where the composition for cleaning tooth surfaces is applied;
removing an excess of the composition for cleaning tooth surfaces after the tooth surfaces are polished, without washing a mouth with water; and
maintaining a state in which a part of the composition for cleaning tooth surfaces remains in the mouth.

2. A composition for cleaning tooth surfaces comprising:
1 to 20% by weight of a casein phosphopeptide-amorphous calcium phosphate complex and/or a casein phosphopeptide-amorphous calcium fluoride phosphate complex;
0.001 to 3% by weight of xylitol and/or sodium saccharin;
20 to 80% by weight of a wetting agent;
0.1 to 20% by weight of silica whose average particle size is no less than 0.5 μm and no more than 50 μm; and
water as a main component of a reminder,
the composition for cleaning tooth surfaces to be applied to tooth surfaces, used for polishing the tooth surfaces, an excess of which is removed after the tooth surfaces are polished without washing a mouth with water, and maintained in a state in which a part of which remains in the mouth.

3. A method for using a composition for cleaning tooth surfaces, the method comprising:
applying a composition for cleaning tooth surfaces to tooth surfaces, the composition comprising:
1 to 20% by weight of a casein phosphopeptide-amorphous calcium phosphate complex and/or a casein phosphopeptide-amorphous calcium fluoride phosphate complex;
0.001 to 3% by weight of xylitol and/or sodium saccharin;
20 to 80% by weight of a wetting agent;
0.1 to 20% by weight of silica whose average particle size is no less than 0.5 μm and no more than 50 μm; and
water as a main component of a reminder;
polishing the tooth surfaces with the composition;
removing an excess of the composition after the tooth surfaces are polished, without washing a mouth with water; and
maintaining the composition in a state in which a part of the composition remains in the mouth.
